(19) United States
(12) Patent Application Publication
(10) Pub. No.: US 2010/0273737 A1
(43) Pub. Date: Oct. 28, 2010

(54) SUPPLEMENTARY FOOD COMPOSITION FOR REDUCING BODY ODOR

(76) Inventors: Jin-Man KIM, Seoul (KR); Duck-Rog Lee, Yongin-si (KR); Ae-Young Song, Seoul (KR)

Correspondence Address: BIRCH STEWART KOLASCH & BIRCH, PO BOX 747, FALLS CHURCH, VA, 22040-0747, US

(21) Appl. No.: 12/829,245

(22) Filed: Jul. 1, 2010

Related U.S. Application Data
(62) Division of application No. 11/597,956, filed on Nov. 28, 2006.

(30) Foreign Application Priority Data
Sep. 14, 2005 (KR) 10-2005-0085512

(51) Int. Cl. A61K 31/722; A61P 43/00

(52) U.S. Cl. 514/55

(57) ABSTRACT

A food supplement for reducing body odor which includes a mixture of edible carriers with chitin aqueous solution lyophilized powder, chitosan aqueous solution lyophilized powder, chitin powder, chitosan powder, or a combination of these ingredients, in the form of tablets or pills. The food supplement has the outstanding effect of reducing various types of body odors, including Koreans' unique body odor, such as Kimchi smell or garlic smell; westerners' unique body odor, such as cheese smell or butter smell, Arabs' unique body odor, such as mutton smell, armpit odor and body odor that is unique to the aged.

This application is a Divisional of co-pending application Ser. No. 11/597,956, filed on Nov. 28, 2006, for which priority is claimed under 35 U.S.C. §120. Application Ser. No. 11/597,956 is the national phase of PCT International Application No. PCT/KR2006/002863 filed on Jul. 20, 2006 under 35 U.S.C. §371, which claims priority from Korean Application No. 10-2005-0085512 filed on Sep. 14, 2005. The entire contents of each of the above-identified applications are hereby incorporated by reference.

### TECHNICAL FIELD

The present invention relates to supplementary food composition for reducing body odor. The composition of the present invention contains two or more ingredients selected from the group consisting of chitin aqueous solution lyophilized powder, chitosan aqueous solution lyophilized powder, chitin powder and chitosan powder. More specifically, edible carriers are mixed with the two or more selected from the group consisting of chitin aqueous solution lyophilized powder, chitosan aqueous solution lyophilized powder, chitin powder and chitosan powder to prepare the supplementary food composition for reducing body odor that is manufactured in the form of tablets or pills.

### BACKGROUND ART

Chitin is an animal fiber widely found in nature, including in the shells of crabs and shrimps; the epidermis or shells of insects, such as beetles and grasshoppers; shellfish, such as clams and oysters; the bones of squids; and the cell walls of fungi, such as mushrooms and yeast. Like fiber in the vegetable kingdom, Chitin is a natural high-polymer substance that plays the role of supporter and protector of the organism.

Chitin is a polysaccharide with a molecular weight of more than 1 million; with β-1,4 linkages to oxygen and it contains at least 5,000 N-ethyl-D glucosamine side chains. While not as abundantly produced in nature as chitin is, chitosan exists in the cell walls of some molds. It is a polysaccharide linked with 2-amino-2-deoxy-D-glucose by β-1,4 linkages. As chitin is deacetylated to produce chitosan, the molecular weight of chitosan is slightly smaller than that of chitin. In order to make chitosan, N-acetyl group has to be taken out of chitin, and this process is called as deacetylation. Chitosan is manufactured through the deacetylation of chitin.

All over the world, disposed crustacean, such as crabs and shrimps amounts to over 144 million tons, and the amount of chitin that is disposed in the marine products manufacturing process increases by 120,000 tons annually. Only 3,000 tons out of this large amount is processed into chitin and chitosan. A large amount of chitin has been disposed and only a small part of it has been limitedly used. However, as studies on chitin and chitosan have recently been actively carried out all over the world including Japan, Korea, China, the United States and European countries where their functions have been newly discovered, they have been used to serve a wider range of purposes. The industrial production of chitin and chitosan started in Japan in 1970, and presently, 1,100 tons are produced annually for industrial purposes. In Korea, about 400 tons are estimated to be produced yearly. In addition, China, the United States, Poland, Norway, Taiwan, Thailand, India and Russia are also chitin and chitosan producers. Chitin itself serves little purpose and is mostly used as a material to make chitosan.

Recent research results indicate that chitin chitosan absorbs and excretes excessive, harmful cholesterol in the human body. It also acts against cancer by inhibiting the proliferation of cancer cells. It also inhibits high blood pressure, helps in the proliferation of useful bacteria inside the intestines and activates cells. It absorbs chloride ions that cause high blood pressure so that it prevents the chloride ions from being absorbed by the intestines and pushes them out of the body instead. It also has other effects such as controlling blood sugar, improving functions of the liver and discharging heavy metal and other pollutants in the body.

In this invention, an edible carrier is mixed with two or more ingredients, selected from the group consisting of chitin aqueous solution lyophilized powder, chitosan aqueous solution lyophilized powder, chitin powder and chitosan powder, and is manufactured in the form of tablets or pills. The invention was completed by confirming its effect of reducing body odor.

Therefore, the primary object of this invention is to provide supplementary food composition that has the effect of reducing body odor.

### DISCLOSURE OF THE INVENTION

### Technical Problem

The aforementioned object of this invention was achieved by manufacturing supplementary food composition in the form of tablets or pills which consist of two or more ingredients selected from the group consisting of chitin aqueous solution lyophilized powder, chitosan aqueous solution lyophilized powder, chitin powder and chitosan powder and an edible carrier, and then confirming its effect of reducing body odor.

### Technical Solution

This invention provides a food supplement for reducing body odor that contains one or more ingredients among chitin aqueous solution lyophilized powder, chitosan aqueous solution lyophilized powder, chitin powder and chitosan powder.

To obtain the chitin aqueous solution lyophilized powder and chitosan aqueous solution lyophilized powder for this invention, chitin aqueous solution and chitosan aqueous solution that can be easily purchased in the market are lyophilized and processed into powder form.

In the present invention, a desirable composition of a supplementary food for reducing body odor is 20˜70 weight percentage of food carriers and 30˜80 weight percentage of one or more ingredients selected from the group consisting of chitin aqueous solution lyophilized powder, chitosan aqueous solution lyophilized powder, chitin powder and chitosan powder.

In the event that the carriers are mixed in more than the above-mentioned composition ratio, the effect of body odor reduction is weakened. On the other hand, the product applications become difficult, and economical efficiency declines when the carriers' weight percentage falls below the recommended ratio, as the effect of mixture with the rest of the ingredients deteriorates.

Any food carrier known to the industry can be used for this invention. Carriers can be chosen appropriately according to the desired forms of the supplementary food, such as tablets or pills. For instance, lactose, starch, Magnesium stearate and Talc can be used as carriers for this invention.

Like Vitamins C or A, the food supplement of this invention can be manufactured in the form of tablets, pills or capsules to be conveniently taken several times a day to remarkably reduce body odor.

### BEST MODE

The followings are detailed methodological descriptions of this invention, whereas the rights of this invention are not limited to the enforcement examples mentioned below.

### Examples

### Manufacturing Supplementary Food Composition for Reducing Body Odor

Chitin aqueous solution and chitosan aqueous solution were purchased, lyophilized and processed into powder, whereas chitin and chitosan powders were purchased to use in this invention.

The ingredients prepared as mentioned above were mixed in the makeup indicated in Table 1, and manufactured into tablets.

TABLE 1
Composition of one tablet of supplementary food for reducing body odor (Unit: mg)

| | Example | Example | Example | Example | Example | Example | Example | Example | Example |
|---|---|---|---|---|---|---|---|---|---|
| Classification | 1 | 2 | 3 | 4 | 5 | 6 | 7 | 8 | 9 |
| Chitin aqueous | 300 | 0 | 0 | 0 | 100 | 100 | 0 | 100 | 50 |
| solution lyophi- | | | | | | | | | |
| lized powder | | | | | | | | | |
| Chitosan aqueous | 0 | 300 | 0 | 0 | 0 | 200 | 100 | 0 | 100 |
| solution lyophi- | | | | | | | | | |
| lized powder | | | | | | | | | |
| Chitin powder | 0 | 0 | 300 | 0 | 200 | 0 | 100 | 100 | 100 |
| Chitosan powder | 0 | 0 | 0 | 300 | 0 | 0 | 100 | 100 | 50 |
| Lactose | 100 | 100 | 100 | 100 | 100 | 100 | 100 | 100 | 100 |
| Starch | 100 | 100 | 100 | 100 | 100 | 100 | 100 | 100 | 100 |
| Talc | 0 | 2 | 0 | 2 | 0 | 2 | 0 | 2 | 0 |
| Magnesium | Proper | Proper | Proper | Proper | Proper | Proper | Proper | Proper | Proper |
| stearate | dose | dose | dose | dose | dose | dose | dose | dose | dose |

### Experiment Example 1

### A Research on Body Odor Reducing Effect Targeting Koreans

Fifty male and female Koreans were selected and divided into ten groups each of which was composed of five people. One of the ten groups was set to be a control group, and each of the other nine groups took two tablets of the supplementary food composition for reducing body odor in Examples 1 to 9, in the mornings and evenings for one month. After one month, the effect of reducing body odors unique to Koreans, such as Kimchi smell and garlic smell was compared. The evaluation of odor reducing effect was made based on a five-point scale (5=Odor reducing effect is the best; 4=Odor reducing effect is quite good; 3=Odor reducing effect is mediocre, 2=Odor reducing effect is small; 1=There is no odor reducing effect). The panel members and ten sensory evaluators were asked to evaluate the effect, and the points were averaged out.

The results of the research on body odor reducing effect are indicated in Table 2 as below.

TABLE 2
Results of the research on body odor
reducing effect targeting Koreans

| | Body odor reducing effect |
|---|---|
| Control group | 1.0 |
| Example 1 | 4.5 |
| Example 2 | 4.6 |
| Example 3 | 4.9 |
| Example 4 | 4.5 |
| Example 5 | 4.8 |
| Example 6 | 4.7 |
| Example 7 | 4.3 |
| Example 8 | 4.6 |
| Example 9 | 5.0 |

Through the results of evaluations by the panel members and sensory evaluators about the body odor reducing effect, it was confirmed that the unique odor often found in Koreans, such as Kimchi smell or garlic smell, was significantly reduced in the Koreans who took the food supplement of this invention that includes chitin aqueous solution lyophilized powder, chitosan aqueous solution lyophilized powder, chitin powder, chitosan powder, or the composition of these ingredients. The same result is seen in Table 2 in the above.

### Experiment Example 2

### A Research on Body Odor Reducing Effect Targeting Westerners

Fifty male and female Europeans who have stayed in Korea for less than three months were selected and divided into ten groups each of which was composed of five people. One of the ten groups was set to be a control group, and each of the other nine groups took two tablets of the food supplement for reducing body odor in Examples 1 to 9, in the mornings and evenings for one month. After one month, the effect of reducing body odors unique to westerners, such as cheese smell and butter smell, was compared. The evaluation of odor reducing effect was made based on a five-point scale (5=Odor reducing effect is the best; 4=Odor reducing effect is quite good; 3=Odor reducing effect is mediocre, 2=Odor reducing effect is small; 1=There is no odor reducing effect). The panel members themselves and ten sensory evaluators were asked to rate the effect, and the points were averaged out.

The results of the research on body odor reducing effect are indicated in Table 3 as below.

TABLE 3
Results of the research on body odor
reducing effect targeting westerners

| | Body odor reducing effect |
|---|---|
| Control group | 1.0 |
| Example 1 | 4.6 |
| Example 2 | 4.9 |
| Example 3 | 4.5 |
| Example 4 | 4.6 |
| Example 5 | 4.7 |
| Example 6 | 4.3 |
| Example 7 | 5.0 |
| Example 8 | 4.9 |
| Example 9 | 4.8 |

Through the results of evaluations by panels and sensory evaluators about body odor reducing effect, it was confirmed that the westerners' unique odor, such as cheese smell or butter smell, was significantly reduced in the westerners who took the food supplement of this invention that includes chitin aqueous solution lyophilized powder, chitosan aqueous solution lyophilized powder, chitin powder, chitosan powder or the composition of these ingredients. The same result is seen in Table 3 in the above.

### Experiment Example 3

### A research on Body Odor Reducing Effect Targeting Arabs

Fifty male and female Arabs from Saudi Arabia and other Arab nations who have stayed in Korea for less than three months were selected and divided into ten groups each of which was composed of five people. One of the ten groups was set to be a control group, and each of the other nine groups took two tablets of the supplementary food composition for reducing body odor in Examples 1 to 9, in the mornings and evenings for one month. After one month, the effect of reducing body odors was compared. The evaluation of odor reducing effect was made based on the five point scale (5=Odor reducing effect is the best; 4=Odor reducing effect is quite good; 3=Odor reducing effect is mediocre, 2=Odor reducing effect is small; 1=There is no odor reducing effect). The panels themselves and ten sensory evaluators were asked to rate the effect, and the points were averaged out.

The results of the research on body odor reducing effect are indicated in Table 4 as below.

TABLE 4
Results of the research on body odor
reducing effect targeting Arabs

| | Body odor reducing effect |
|---|---|
| Control group | 1.0 |
| Example 1 | 4.5 |
| Example 2 | 4.3 |
| Example 3 | 5.0 |
| Example 4 | 4.4 |
| Example 5 | 4.9 |
| Example 6 | 4.6 |
| Example 7 | 4.8 |
| Example 8 | 5.0 |
| Example 9 | 4.9 |

Through the results of evaluations by panels and sensory evaluators about body odor reducing effect, it was confirmed that the Arabs' unique odor, such as mutton smell, was significantly reduced in the Arabs who took the food supplement of this invention that includes chitin aqueous solution lyophilized powder, chitosan aqueous solution lyophilized powder, chitin powder, chitosan powder, or the composition of these ingredients. The same result is seen in Table 4 in the above.

### Experiment Example 4

### A Research on Armpit Odor Reducing Effect

Fifty male and female adults were selected and divided into ten groups each of which was composed of five people. One of the ten groups was set to be a control group, and each of the other nine groups took two tablets of the supplementary food composition for reducing body odor in Examples 1 to 9, in the mornings and evenings for one month. After one month, the effect of reducing armpit odors was compared. The evaluation of odor reducing effect was made based on the five point scale (5=Odor reducing effect is the best; 4=Odor reducing effect is quite good; 3=Odor reducing effect is mediocre, 2=Odor reducing effect is small; 1=There is no odor reducing effect). The panels themselves and ten sensory evaluators were asked to rate the effect, and the points were averaged out.

The results of the research on body odor reducing effect are indicated in Table 5 as below.

TABLE 5
Results of the research on armpit odor reducing effect

| | Armpit odor reducing effect |
|---|---|
| Control group | 1.0 |
| Example 1 | 4.3 |
| Example 2 | 4.8 |
| Example 3 | 4.6 |
| Example 4 | 4.9 |
| Example 5 | 5.0 |
| Example 6 | 4.4 |
| Example 7 | 4.8 |
| Example 8 | 4.6 |
| Example 9 | 4.7 |

### Experiment Example 5

### A Research on Effect to Reduce Body Odor Unique in Elderly People

Fifty males and females aged 70 or older were selected and divided into ten groups each of which was composed of five people. One of the ten groups was set to be a control group, and each of the other nine groups took two tablets of the supplementary food composition for reducing body odor in Examples 1 to 9, in the mornings and evenings for one month. After one month, the body odor reducing effect was compared. The evaluation of odor reducing effect was made based on the five point scale (5=Odor reducing effect is the best; 4=Odor reducing effect is quite good; 3=Odor reducing effect is mediocre, 2=Odor reducing effect is small; 1=There is no odor reducing effect). The panels themselves and ten sensory evaluators were asked to rate the effect, and the points were averaged out.

The results of the research on body odor reducing effect are indicated in Table 6 as below.

TABLE 6
Results of the research on effect to reduce
body odor unique in elderly people

| | Body odor reducing effect |
|---|---|
| Control group | 1.0 |
| Example 1 | 4.3 |
| Example 2 | 4.9 |
| Example 3 | 4.2 |
| Example 4 | 4.6 |
| Example 5 | 4.0 |
| Example 6 | 4.6 |
| Example 7 | 4.8 |
| Example 8 | 5.0 |
| Example 9 | 4.3 |

### INDUSTRIAL APPLICABILITY

As explained above, this invention, that is a mixture of edible carriers with chitin aqueous solution lyophilized powder, chitosan aqueous solution lyophilized powder, chitin powder, chitosan powder, or the composition of these ingredients and that comes in the forms of tablets or pills, has an excellent effect in reducing various types of body odors, including Koreans' unique body odor, such as Kimchi smell or garlic smell, Europeans' unique body odor, such as cheese smell or butter smell, Arabs' unique body odor, such as mutton smell, armpit odor and body odor that is unique in the aged. For this reason, this supplementary food is expected to be a highly innovative and useful invention in the food industry.

1. A method of reducing body odor in humans which comprises administering a supplementary food composition in the form of tablets, pills or capsules which consists essentially of a pharmaceutical acceptable amount of one or more active ingredients selected from the group consisting of chitin aqueous solution lyophilized powder, chitosan aqueous solution lyophilized powder, chitin powder and chitosan powder.

2. The supplementary food composition of claim 1, further containing an edible food carrier.

3. The supplementary food composition of claim 2, wherein the active ingredient is present in an amount of 30 to 80% by weight and the edible food carrier is present in an amount of 20-70% by weight.

4. The supplementary food composition of claim 2, wherein the edible food carrier is lactose, starch, magnesium stearate and tale.
